(19) United States
(12) Patent Application Publication
(10) Pub. No.: US 2026/0218156 A1
(43) Pub. Date: Jul. 30, 2026

(54) UROPATHOGENIC ESCHERICHIA COLI RECOMBINANT PROTEIN COMPOSITION LS, CONSTRUCTION, EXPRESSION, AND PURIFICATION METHOD THEREFOR, AND USE THEREOF

(71) Applicant: Zhejiang Chinese Medical University, Zhejiang (CN)

(72) Inventors: Aisong ZHU, Zhejiang (CN); Hongxia NIU, Zhejiang (CN); Quanjie HU, Zhejiang (CN); Zhuoxuan LV, Zhejiang (CN); Shuai ZHOU, Zhejiang (CN)

(73) Assignee: Zhejiang Chinese Medical University, Zhejiang (CN)

(21) Appl. No.: 19/140,351

(22) PCT Filed: Jan. 24, 2024
(86) PCT No.: PCT/CN2024/073775

§ 371 Date: Jun. 17, 2025

(30) Foreign Application Priority Data
Jul. 19, 2023 (CN) 202310885454.0

(51) Int. Cl. C12N 9/90; A61K 39/108; A61P 37/04; B01D 15/38; C12N 15/70

(52) U.S. Cl. CPC C12N 9/90; A61K 39/0258; A61P 37/04; B01D 15/3828; C12N 15/70; C12Y 502/01008; C12Y 503/01028

(57) ABSTRACT

A uropathogenic E. coli recombinant protein composition LS, a construction, expression, and purification method therefor, and a use thereof are belong to the technical field of genetic engineering. Performing PCR amplification on LpcA and SurA genes, and respectively inserting the LpcA and SurA genes into multiple cloning sites of a cloning vector to construct recombinant vectors; followed by transforming the recombinant vectors into E. coli for protein expression; and finally performing purification to obtain the composition LS of recombinant proteins LpcA and SurA applicable in urinary tract infection vaccines. The advantages are: the protein vaccine can induce stronger cellular and humoral immune responses in animals; and animal challenge experiments demonstrate its protective efficacy in reducing bacterial loads in the bladder and kidneys.

### TECHNICAL FIELD

The present invention relates to the technical field of genetic engineering, and in particular, to a uropathogenic E. coli recombinant protein composition LS, a construction, expression, and purification method therefor, and a use thereof.

### BACKGROUND OF RELATED ART

Urinary tract infection (UTI) is the second most common infectious disease that seriously threatens human health, with uropathogenic E. coli (UPEC) being its primary pathogen. It is reported that 60% of women and 12% of men experience at least one UTI episode in their lifetime, while 20%-30% of female patients suffer a recurrence within 3-4 months after the first episode. Some patients experience multiple recurrences and may even be infected throughout their lives. Postmenopausal women, pregnant women, children, and patients with immune dysfunction are particularly susceptible to UTIs. Additionally, factors such as indwelling catheters and anatomical abnormalities of the urinary tract can increase the incidence of UTIs. UTIs severely impact patients' quality of life and may even lead to life-threatening urosepsis. Preventing and reducing UTI recurrence remains one of the major challenges in this field of research.

Although clinical antibiotic therapy can alleviate acute UTIs, its effectiveness in preventing and treating recurrent UTIs is limited. Moreover, the overuse of antibiotics not only increases the risk of bacterial resistance, leading to treatment failure, but may also contribute to the development of refractory UTIs. In recent years, UTI vaccines have emerged as a research hotspot for preventing and treating recurrent UTIs, as they can stimulate antigen-specific immune responses in the body, offering dual protective and therapeutic effects. Depending on their components and preparation methods, UTI vaccines can be classified into three main types: whole-cell/lysate vaccines, live-attenuated vaccines, and subunit vaccines. Among these, whole-cell/lysate vaccines have already been marketed in Europe and, although they offer a new approach to clinical treatment and prevention of recurrent UTIs, they require prolonged and consecutive administration to achieve optimal efficacy; live-attenuated vaccines induce protective immune responses of limited duration, resulting in relatively fewer studies on this type; and protein subunit vaccines, as a novel vaccine type constructed by combining bacterial protective protein antigens with delivery adjuvants, have become a research hotspot in UTI vaccines due to their well-defined composition, high safety, and capability of inducing strong immune memory responses to provide long-lasting protection.

Sur A is a parvulin-like peptidyl-prolyl isomerase (PPIase) that functions as a molecular chaperone in the periplasm (the space between the inner and outer membranes) of E. coli, participating in the assembly, transport, and folding of outer membrane proteins. LpcA (also known as GmhA) is a cytoplasmic phosphoheptose isomerase involved in synthesizing the core component of triglycerides as lipopolysaccharide (LPS) precursors. Previous studies have found that deleting the genes encoding LpcA and SurA in UPEC significantly reduces bacterial survival and persistent infection ability in mice. Therefore, we infer that these two proteins may serve as candidate targets for both drug development and vaccine research against UPEC-induced UTIs. However, obtaining these two proteins through genetic engineering methods and validating their use in UTI vaccines remains an unresolved technical challenge.

### SUMMARY OF THE INVENTION

In view of the problems in the prior art, the present invention is intended to design and provide technical solutions for a uropathogenic E. coli recombinant protein composition LS, a construction, expression, and purification method therefor, and a use thereof.

The objectives of the present invention are achieved by the following technical solutions:

In a first aspect, the present invention provides a uropathogenic E. coli recombinant protein composition LS, containing recombinant proteins LpcA and SurA, where the recombinant protein LpcA has an amino acid sequence shown in SEQ ID NO: 2, and the recombinant protein SurA has an amino acid sequence shown in SEQ ID NO: 4.

Furthermore, a mass ratio of the recombinant protein LpcA to the recombinant protein SurA is 1:1.

In a second aspect, the present invention provides a use of the uropathogenic E. coli recombinant protein composition LS described above for preparing a drug for treating uropathogenic E. coli-induced urinary tract infections.

In a third aspect, the present invention provides a use of the uropathogenic E. coli recombinant protein composition LS described above for preparing a vaccine for preventing uropathogenic E. coli-induced urinary tract infections.

In a fourth aspect, the present invention provides a construction, expression, and purification method for the uropathogenic E. coli recombinant protein composition LS described above, including the following steps:
1) performing PCR amplification on LpcA and SurA genes, and respectively inserting the LpcA and SurA genes into multiple cloning sites of a cloning vector pET30a(+) to construct recombinant vectors pET30a(+)-LpcA and pET30a(+)-SurA;
2) transforming the recombinant vectors into E. coli to obtain a strain expressing the recombinant proteins LpcA and SurA, and utilizing the strain to express the recombinant proteins LpcA and SurA; and
3) purifying the recombinant proteins LpcA and SurA.

Furthermore, the step 1) specifically includes:
(1) based on gene sequences of LpcA (GeneID: 949134, 579 bp) and SurA (GeneID: 944812, 1287 bp) from E. coli str. K12 in GenBank, along with restriction sites of the cloning vector, designing upstream and downstream primers containing endonucleases: SurA F and SurA R as well as LpcA F and LpcA R respectively, with nucleotide sequences of the SurA R, the Sur A R, the LpcA F, and the LpcA R shown in SEQ ID NO: 5, SEQ ID NO: 6, SEQ ID NO: 7, and SEQ ID NO: 8 respectively;
(2) extracting DNA from a uropathogenic E. coli strain UTI89 as a template, performing the PCR amplification on the gene sequences of LpcA and SurA using the primers obtained in the step (1), and purifying LpcA and Sur A PCR amplification products by gel extraction for later use;
(3) extracting a pET30a(+) plasmid: amplifying an E. coli strain containing the pET30a(+) plasmid to 5 mL, and extracting the pET30a(+) plasmid using a plasmid extraction kit; and
(4) digesting the LpcA and SurA PCR amplification products obtained from the step (2) and the pET30a(+) plasmid extracted from the step (3) with EcoRI and HindIII restriction endonucleases; purifying digested LpcA and SurA PCR amplification products and digested pET30a(+) plasmid through PCR product purification and gel extraction; ligating purified LpcA gene and pET30a(+) plasmid as well as purified SurA gene and pET30a(+) plasmid using T4 DNA ligase; and transforming ligation products into E. coli DH5a, picking positive clones through antibiotic plate screening, extracting plasmids, and performing sequencing identification to obtain the recombinant vectors pET30a(+)-LpcA and pET30a(+)-SurA.

Furthermore, the step 2) specifically includes:
(a) transforming the successfully constructed plasmid vectors pET30a(+)-LpcA and pET30a(+)-SurA respectively into an E. coli expression strain BL21 (DE3) to obtain a recombinant protein-preserving strain;
(b) subjecting the recombinant protein-preserving strain obtained in the step (a) to activation, shaking amplification culture, induction-shaking culture, and centrifugation in sequence to obtain a bacterial cell of the recombinant protein-preserving strain; and
(c) resuspending the bacterial cell obtained in the step (b) in PB buffer, performing sonication in an ice bath and centrifugation, collecting a supernatant and a pellet separately, and analyzing the supernatant and the pellet separately by polyacrylamide gel electrophoresis to assess protein expression profiles.

Furthermore, the step 3) specifically includes:
(A) performing large-scale amplification on the strain expressing the recombinant proteins LpcA and SurA, resuspending a harvested bacterial cell in PB buffer, performing sonication in an ice bath and centrifugation, collecting a supernatant containing the recombinant proteins LpcA and SurA, and performing sterilization by filtration for later use; and
(B) taking purified LpcA and SurA from the supernatant obtained in the step (A) for affinity chromatography and elution to obtain the recombinant proteins LpcA and SurA.

Furthermore, the affinity chromatography and the elution in the step (B) specifically include: first, rinsing an empty chromatography column with sterile water to ensure a smooth flow, adding 1 mL of nickel-charged metal affinity chromatography medium (Ni-NTA) along an inner wall of the column, and after equilibrating the medium with 10 mL of imidazole binding buffer, loading the supernatant containing the recombinant proteins LpcA and Sur A onto the medium for binding; then, washing the column with 10 mL of imidazole binding buffer and 10 mL of imidazole wash buffer in sequence, and performing gradient elution using 50 mM, 100 mM, 200 mM, and 300 mM imidazole elution buffers; and finally, performing elution using 200-300 mM imidazole elution buffer to obtain the recombinant proteins LpcA and SurA respectively, and after protein quantification, storing the recombinant proteins LpcA and SurA for later use.

The present invention has the following beneficial effects: the present invention successfully constructs a strain expressing proteins LpcA and SurA and establishes a chromatography purification method for the proteins LpcA and SurA using genetic engineering technology. A combination of the proteins LpcA and SurA can induce stronger cellular and humoral immune responses, has a certain protective effect against uropathogenic E. coli in animals, and can be used in the development of anti-uropathogenic E. coli vaccines.

### BRIEF DESCRIPTION OF THE DRAWING

FIG. 1 shows expression and purification results of recombinant proteins LpcA and SurA: (A) shows the expression results of the recombinant proteins LpcA and SurA, where Lane M: marker; Lane 1: LpcA-expressing cell lysate; Lane 2: supernatant of the LpcA-expressing cell lysate; Lane 3: pellet of the LpcA-expressing cell lysate; Lane 4: Sur A-expressing cell lysate; Lane 5: supernatant of the SurA-expressing cell lysate; and Lane 6: pellet of the SurA-expressing cell lysate; (B) shows the purification results of the recombinant proteins LpcA and SurA, where Lane M: marker; Lanes 1-2: purified LpcA; and Lanes 3-4: purified SurA;

FIG. 2 shows antigen-specific IFN-γ, IL-2, and IL-17 levels induced by a subunit vaccine LS/DP at 4 weeks post-final immunization: (A) shows LpcA-specific and Sur A-specific IFN-γ levels; (B) shows LpcA-specific and Sur A-specific IL-2 levels; and (C) shows LpcA-specific and Sur A-specific IL-17 levels;

FIG. 3 shows antigen-specific IFN-γ, IL-2, and IL-17 levels induced by a subunit vaccine LS/DP at 8 weeks post-final immunization: (A) shows LpcA-specific IFN-γ levels; (B) shows SurA-specific IFN-γ levels; (C) shows LpcA-specific IL-2 levels; (D) shows SurA-specific IL-2 levels; (E) shows LpcA-specific IL-17 levels; and (F) shows Sur A-specific IL-17 levels;

FIG. 4 shows antigen-specific IgG and IgG2c antibody levels induced by a subunit vaccine LS/DDA-PolyI:C at 4 weeks post-final immunization: (A) shows LpcA-specific IgG levels; (B) shows LpcA-specific IgG2c levels; (C) shows SurA-specific IgG levels; and (D) shows SurA-specific IgG2c levels;

FIG. 5 shows antigen-specific IgG and IgG2c antibody levels induced by a subunit vaccine LS/DDA-PolyI:C at 8 weeks post-final immunization: (A) shows LpcA-specific IgG levels; (B) shows LpcA-specific IgG2c levels; (C) shows SurA-specific IgG levels; and (D) shows SurA-specific IgG2c levels;

FIG. 6 shows a protective effect of a subunit vaccine LS/DDA-PolyI:C in mice; and

FIG. 7 is a construction diagram of recombinant gene vectors pET30a(+)-LpcA and pET30a(+)-SurA.

### DETAILED DESCRIPTION OF THE EMBODIMENTS

The present invention will be further described with reference to embodiments.

### Example 1: Construction, Expression, and Purification of a Uropathogenic E. coli Recombinant Protein Composition LS

First, plasmid vectors pET30a(+)-LpcA and pET30a(+)-SurA were constructed separately. FIG. 7 is a construction diagram of recombinant gene vectors pET30a(+)-LpcA and pET30a(+)-SurA; subsequently, successfully constructed plasmid vectors were transformed into E. coli BL21 (DE3) for expressing recombinant proteins LpcA and SurA; then, the recombinant proteins were purified by affinity chromatography using their His-tags; and finally, purified recombinant proteins LpcA and SurA were mixed at a ratio of 1:1 to construct the recombinant protein composition LS.

Specific steps are as follows:

1. Construction of the Recombinant Gene Vector pET30a(+)-LpcA
(1) Based on the gene sequence of LpcA (GeneID: 949134, 579 bp) from E. coli str. K12 in GenBank and restriction sites of the cloning vector, upstream and downstream primers were designed as follows:
upstream primer LpcA F:
5′-CGCGGATCCATGTACCAGGATCTTATTCG-3′ (the italicized and underlined segment represents a BamHI restriction site), with its nucleotide sequence shown in SEQ ID NO: 5;
downstream primer LpcA R:
5′-CCGGAATTCCTTAACCATCTCTTTTTC-3′ (the italicized and underlined segment represents an EcoRI restriction site), with its nucleotide sequence shown in SEQ ID NO: 6.
(2) PCR amplification of the LpcA gene: DNA of uropathogenic E. coli was extracted as a template, the LpcA gene sequence was subjected to PCR amplification using the aforementioned primers, and the PCR amplification product was purified by gel extraction for later use.
(3) Construction of the recombinant vector pET30a(+)-LpcA: the LpcA PCR amplification product and the pET30a(+) plasmid were digested with BamHI and EcoRI restriction endonucleases, respectively; the digested LpcA PCR amplification product and pET30a(+) plasmid were purified through PCR product purification and gel extraction; the purified LpcA gene and pET30a(+) plasmid were ligated using T4 DNA ligase; the ligation product was then transformed into E. coli DH5a; and positive clones were picked through antibiotic plate screening, followed by plasmid extraction and sequencing identification.
(4) Determination of identification results: the LpcA gene sequence in the plasmid extracted from the recombinant E. coli strain DH5a contained no mutations and matched the theoretical sequence in GenBank, with the nucleotide sequence of the LpcA gene shown in SEQ ID NO: 1.
   2. Construction of the Recombinant Gene Vector pET30a(+)-SurA
(1) Based on the gene sequence of SurA (GeneID: 944812, 1287 bp) from E. coli str. K12 in GenBank and restriction sites of the cloning vector, upstream and downstream primers were designed as follows:
upstream primer SurA F:
5′-CGTGAATTCATGAAGAACTGGAAAACGCTGCTTC-3′ (the italicized and underlined segment represents an EcoR I restriction site), with its nucleotide sequence shown in SEQ ID NO: 7;
downstream primer Sur A R:
5′-TCGAAGCTTAGTTGCTCAGGATTTTAACGTAG-3′ (the italicized and underlined segment represents a Hind III restriction site), with its nucleotide sequence shown in SEQ ID NO: 8.
(2) PCR amplification of the SurA gene: DNA from a uropathogenic E. coli strain UTI89 was extracted as a template, the SurA gene sequence was subjected to PCR amplification using the aforementioned primers, and the PCR amplification product was purified by gel extraction for later use.
(3) Extraction of the pET30a(+) plasmid: an E. coli strain containing the pET30a(+) plasmid was amplified to 5 mL, and the pET30a(+) plasmid was extracted using a plasmid extraction kit.
(4) Construction of the recombinant vector pET30a(+)-SurA: the SurA PCR amplification product and the pET30a(+) plasmid were digested with EcoRI and HindIII restriction endonucleases, respectively; the digested SurA PCR amplification product and pET30a(+) plasmid were purified through PCR product purification and gel extraction; the purified SurA gene and pET30a(+) plasmid were ligated using T4 DNA ligase; the ligation product was then transformed into E. coli DH5a; and positive clones were picked through antibiotic plate screening, followed by plasmid extraction and sequencing identification.
(5) Determination of identification results: the SurA gene sequence in the plasmid extracted from the recombinant E. coli strain DH5a contained no mutations and matched the theoretical sequence in GenBank, with the nucleotide sequence of the SurA gene shown in SEQ ID NO: 3.

### 3. Expression of the Recombinant Proteins LpcA (192Aa) and SurA (428Aa)

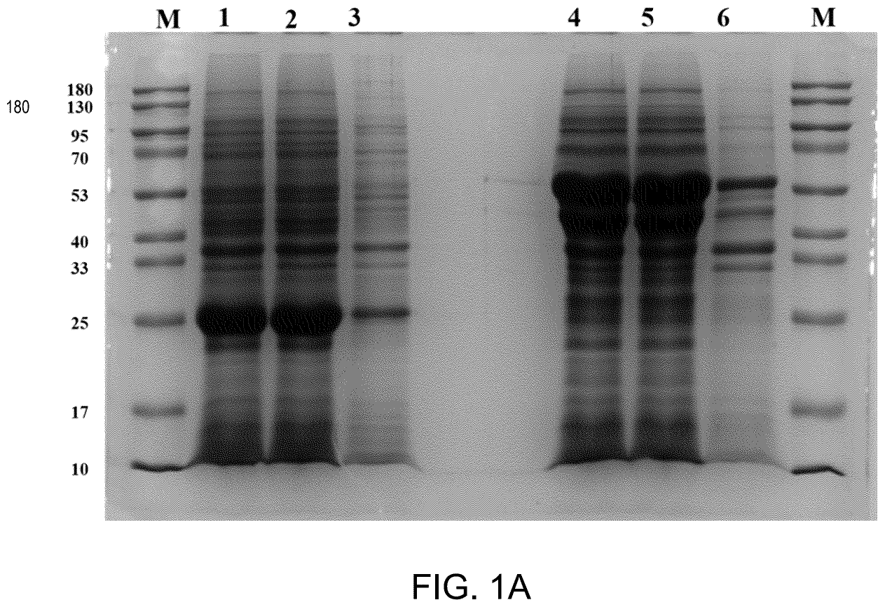

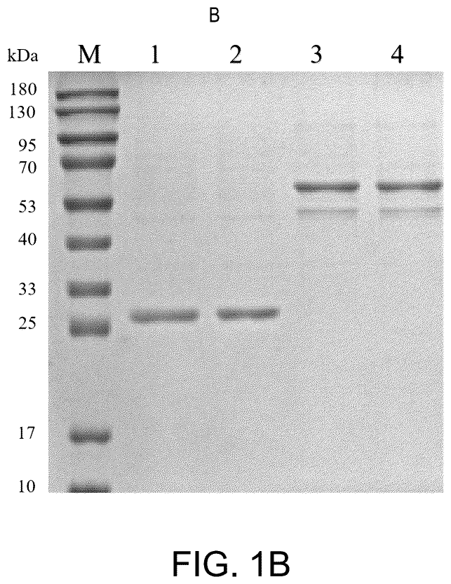

(1) The successfully constructed plasmid vectors pET30a(+)-LpcA and pET30a(+)-Sur A were transformed respectively into an E. coli expression strain BL21 (DE3); positive clones were screened by PCR verification and subjected to sequencing identification; and an identified suitable strain was preserved in 80% glycerol as a recombinant protein-preserving strain.
(2) First, 10 μL of the recombinant protein-preserving strain was inoculated into 5 mL of LB liquid medium for activation; then, 1 mL of activated strain was transferred to 200 mL of LB liquid medium and incubated with shaking until reaching an OD600 of 0.6; subsequently, 100 μL of IPTG (1.0 mmol/L) was added for induction and shaking incubation for 12 h at 25° C.; and finally, bacterial cells were collected by centrifugation at 10,000 rpm for 10 min at 4° C.
(3) The bacterial cells were resuspended in PB buffer (20 mM Na₂HPO₄·12H₂O, 20 mM NaH₂PO₄·2H₂O, pH 7.4) at a ratio of 10 mL per gram of wet cell weight; sonication was performed in an ice bath for 1 h (180-200 W, pulse duration for 4 s followed by an interval of 5 s); and after centrifugation at 10,000 RPM/min for 20 min, a supernatant and a pellet were collected separately and then analyzed separately by polyacrylamide gel electrophoresis to assess protein expression profiles.
(4) The results (as shown in FIG. 1A) demonstrated that the LpcA-expressing strain exhibited a distinct specific protein band at a molecular weight of 17 kDa, while the SurA-expressing strain exhibited a distinct specific protein band at a molecular weight of about 29 kDa. In both cases, the protein was predominantly present in the supernatant, with relatively less protein observed in the pellet.

### 4. Purification of the Recombinant Proteins LpcA and Sur A

(1) Large-scale amplification was performed on the strain expressing the fusion proteins LpcA and SurA; a harvested bacterial cell was resuspended in 20 mM PB buffer and subjected to sonication in an ice bath for about 1 h, followed by centrifugation at 10,000 rpm for 10 min at 4° C.; and a supernatant containing the recombinant proteins LpcA and SurA was collected, filtered through a 0.45 μm membrane for sterilization, and stored for later use.
(2) First, an empty chromatography column was rinsed with sterile water to ensure a smooth flow, 1 mL of nickel-charged metal affinity chromatography medium (Ni-NTA) was added along an inner wall of the column, and after equilibrating the medium with 10 mL of imidazole binding buffer, the supernatant containing the recombinant proteins LpcA and SurA was loaded onto the medium for binding; then, the column was washed with 10 mL of imidazole binding buffer and 10 mL of imidazole wash buffer in sequence, and gradient elution was performed using 50 mM, 100 mM, 200 mM, and 300 mM imidazole elution buffers; and finally, elution concentrations for the target proteins were determined based on sodium dodecyl sulfate-polyacrylamide gel electrophoresis (SDS-PAGE) results.
(3) The results demonstrated that the recombinant protein LpcA was eluted using 200-300 mM imidazole elution buffer while the recombinant protein SurA was eluted with 200-300 mM imidazole elution buffer. Following purification by affinity chromatography, both the recombinant proteins were obtained with a purity of >90%. The purification results of the recombinant proteins LpcA and SurA are shown in FIG. 1B. The purified recombinant proteins LpcA and Sur A were mixed at a mass ratio of 1:1 to construct the recombinant protein composition LS. It was confirmed by sequencing analysis that the recombinant protein LpcA had an amino acid sequence shown in SEQ ID NO: 2 while the recombinant protein Sur A had an amino acid sequence shown in SEQ ID NO: 4.

### Example 2: Preparation of Subunit Vaccines LS/DDA-PolyI:C (LS/DP), L/DP, and S/DP

The recombinant protein composition LS and individual proteins L and S obtained in Example 1 were diluted to 0.4 mg/mL using PBS (phosphate-buffered saline); PolyI:C was dissolved in PBS to a concentration of 0.5 mg/mL; and cationic lipid dimo-thylidioctyl ammonium bromide (DDA) was prepared in sterile distilled water to a concentration of 2.5 mg/mL, heated in a water bath at 80° C. for 10 min, and then cooled to room temperature. 50 μL of PolyI:C solution was thoroughly mixed with an equal volume of protein solution, followed by incubation at room temperature for 1 min. Subsequently, 100 μL of DDA solution was added dropwise to the mixed solution, followed by thorough emulsification to form a homogeneous milky emulsification, thereby obtaining the subunit vaccines LS/DP, L/DP, and S/DP. For immunization, the dose of the vaccines was 200 μL/mouse.

### Example 3: Evaluation of Immunological Activity of the Subunit Vaccines LS/DP, L/DP, and S/DP

1. Experimental materials: subunit vaccine LS/DDA-PolyI:C obtained in Example 1; and phosphate buffered saline (PBS).
2. Experimental animals: C57BL/6 mice.
3. Grouping of experimental animals (three groups in total):
(1) PBS (negative control); (2) LS/DP; (3) L/DP; (4) S/DP; and (5) DDA-PolyI:C (DP adjuvant control).
4. Immunization of animals:

At week 0, the experimental group received a single subcutaneous inguinal immunization with the prepared subunit vaccine (200 μL per animal), and control groups were immunized simultaneously. Following the initial immunization at week 0, the experimental group received two booster immunizations at weeks 2 and 4 using the same dose.
5. Determination of immunological parameters:
(1) Determination of antigen-specific IFN-γ, IL-2, and IL-17 levels in mouse spleen by ELISA

At weeks 4 and 8 post-final immunization, splenic lymphocytes were aseptically isolated from the mice. The secretion levels of IFN-γ, IL-2, and IL-17 by these splenic lymphocytes in response to LpcA and SurA stimulation were determined using enzyme-linked immunosorbent assay (ELISA).

Procedure: the spleen was aseptically removed, homogenized, and filtered through a 200-mesh nylon screen; lymphocytes were isolated using a lymphocyte separation medium; the isolated lymphocytes were inoculated onto a 24-well plate (5×10⁶ cells/well) and stimulated with proteins LpcA and SurA (final concentration: 5 μg/mL for each) respectively; and the cells and the proteins were co-incubated in a 5% CO₂ incubator at 37° C. for 72 h. After incubation, cell culture supernatants were collected and cytokine secretion levels were quantitatively analyzed by ELISA.

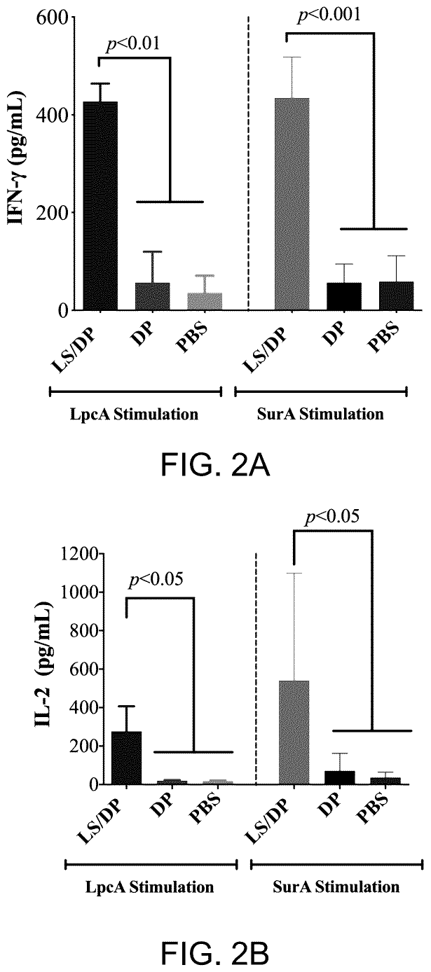

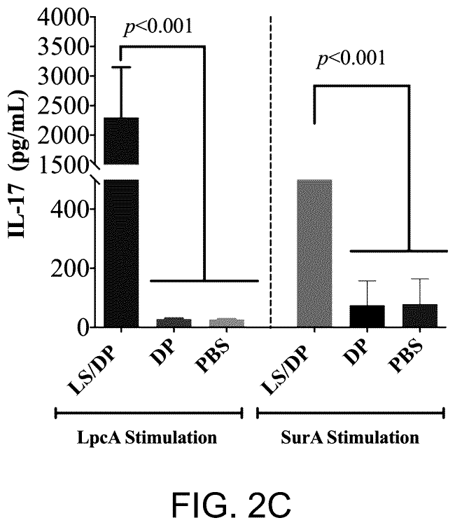

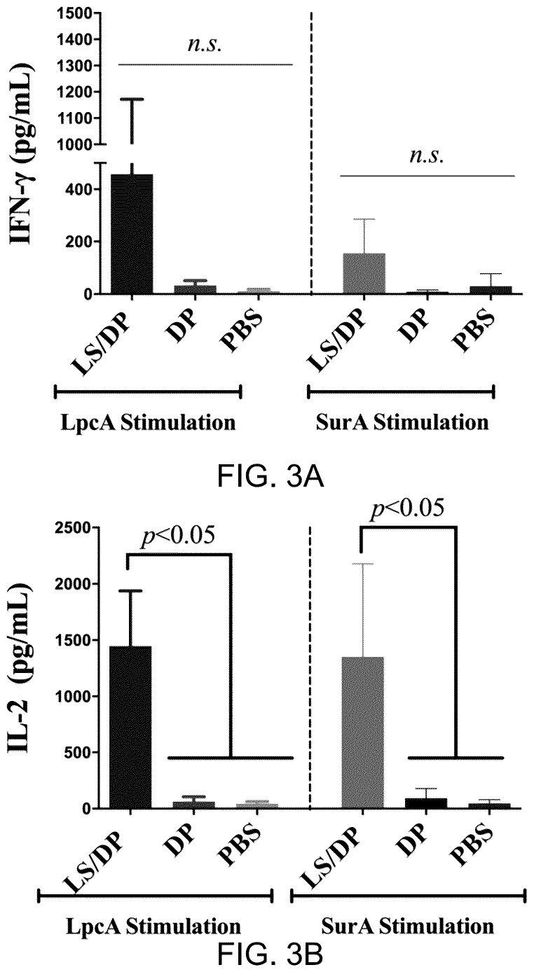

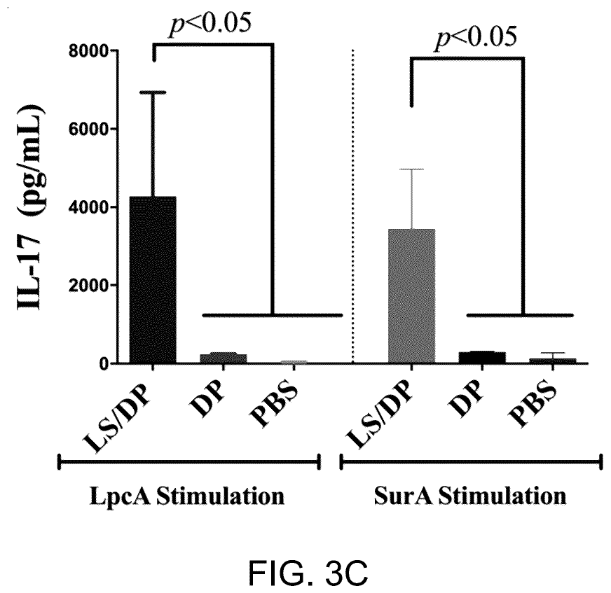

Results: the subunit vaccine LS/DDA-PolyI:C of the present invention induced significantly higher levels of antigen-specific IFN-γ, IL-2, and IL-17 production in splenocytes from immunized mice compared to both PBS and DP control groups, as shown in FIGS. 2 and 3. These results demonstrate that the subunit vaccine LS/DP can induce a high level of (Th1-, Th2-, and Th17-type) cellular immune response in mice.
(2) Determination of antigen-specific antibody levels (IgG and IgG2c) in mice by ELISA

At weeks 4 and 8 post-final immunization, peripheral blood was collected via orbital sampling from the mice, serum was isolated, and LpcA- and Sur A-specific IgG and IgG2c antibody levels in the serum were then determined by enzyme-linked immunosorbent assay (ELISA).

Procedure: a 96-well plate was coated with LpcA (5 μg/mL) and SurA (5 μg/mL) respectively at 100 μL/well and incubated overnight at 4° C.; the plate was washed three times with PBST (300 μL/well, 1 min per wash); and the serum samples were subjected to serial dilutions starting at 1:400 up to 1:20,480, followed by incubation of diluted gradient serum samples in the coated 96-well plate at 37° C. for 1 h. After washing the plate as described above, 200 μL of 1:50,000-diluted rabbit anti-mouse IgG1 or 1:50,000-diluted rabbit anti-mouse IgG2c was added per well, followed by incubation at 37° C. for 1 h. After washing the plate, 100 μL of TMB substrate solution was added per well, followed by a reaction protected from light at room temperature for 15 min; then, the reaction was stopped by adding 100 μL of stop solution (2 N H₂SO₄) per well; and the optical density (OD) value was detected at 450 nm.

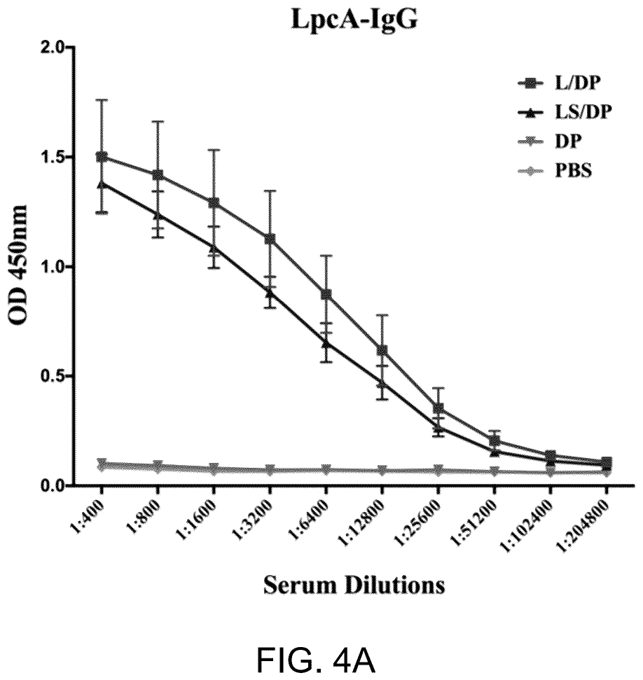

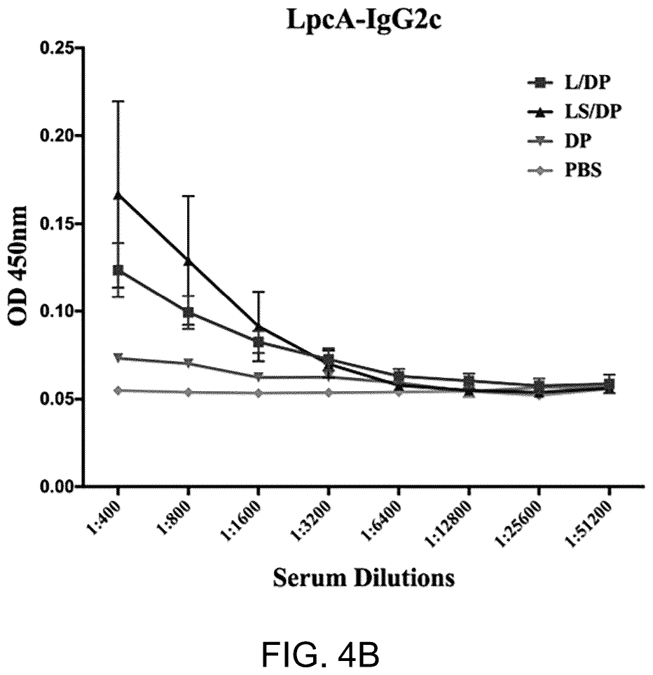

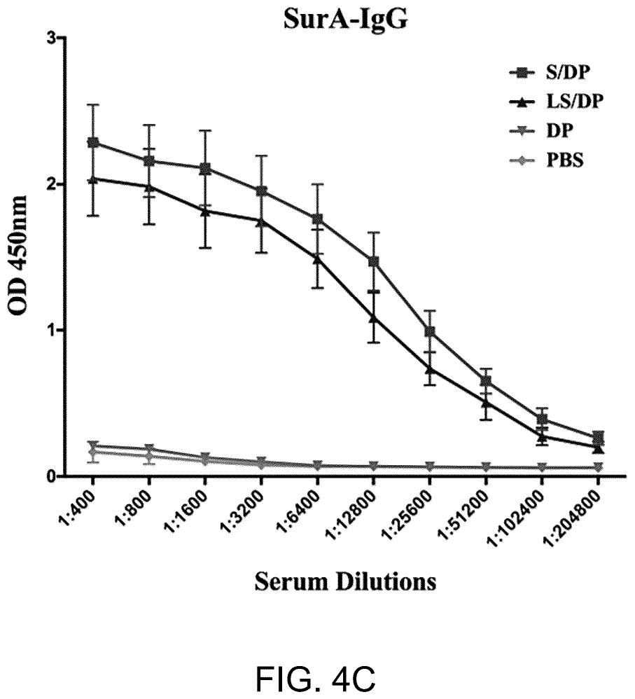

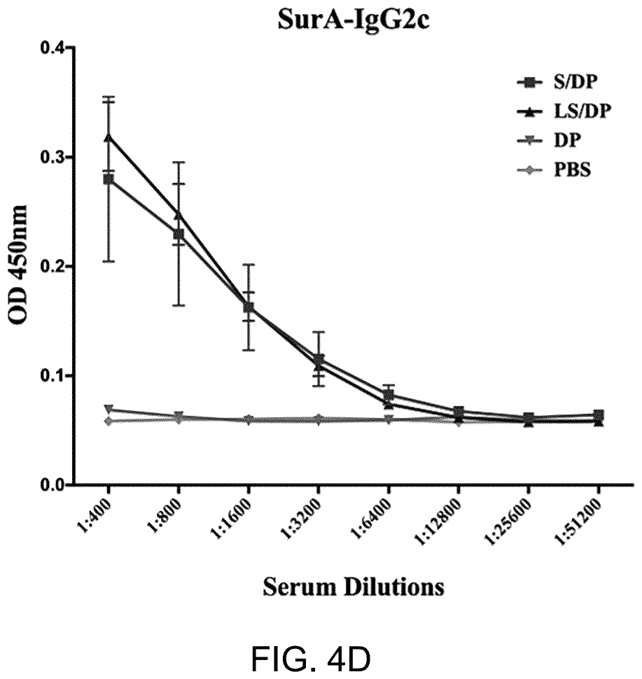

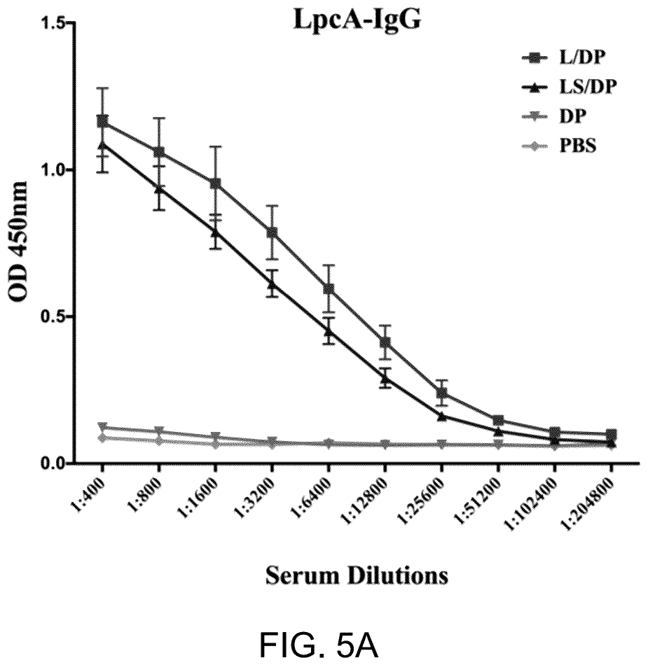

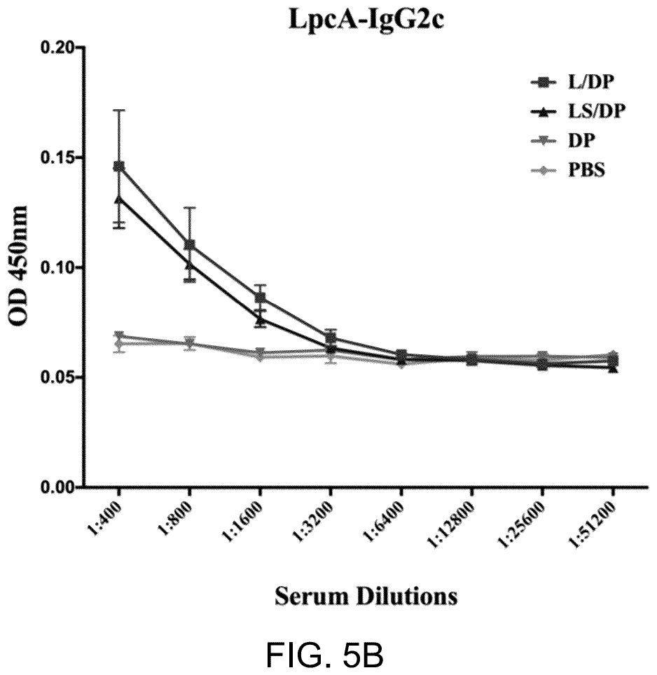

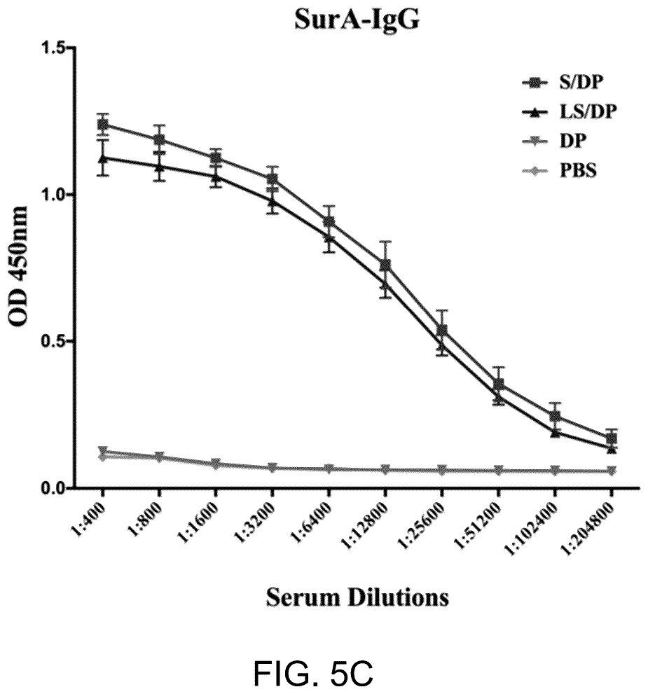

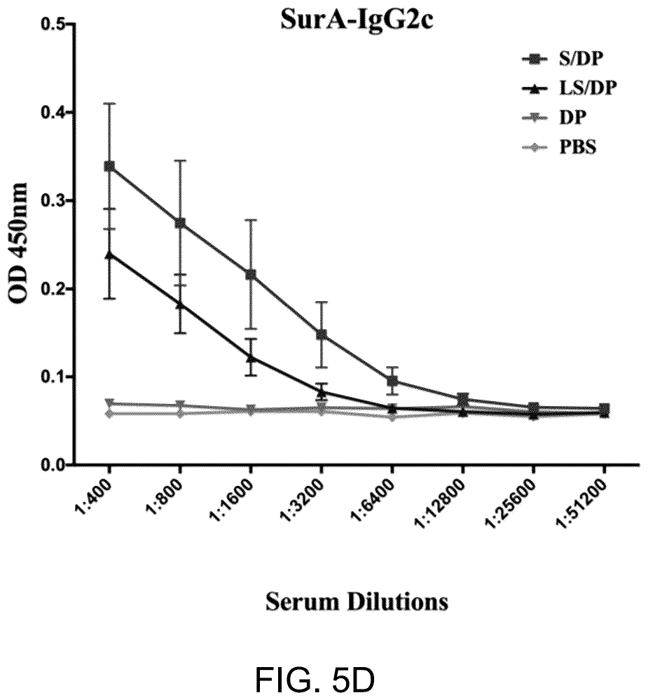

Results: the subunit vaccine LS/DP of the present invention induced significantly higher levels of antibody production in immunized mice compared to both PBS and DDA-PolyI:C control groups, as shown in FIGS. 4 and 5. These results demonstrate that the subunit vaccine LS/DP can induce a high level of humoral immune response in mice.

### Example 4: Evaluation of Protective Effect of the Subunit Vaccine LS/DP

1. Experimental materials: subunit vaccines LS/DP, L/DP, and S/DP; and phosphate buffered saline (PBS).
2. Experimental animals: C57BL/6 mice.
3. Grouping of experimental animals (five groups in total):
(1) PBS (negative control); (2) LS/DP; (3) L/DP; (4) S/DP; and (5) DP (adjuvant control).
4. The animal immunization procedure and time points were the same as those described in Example 3.
5. Evaluation of protective effect:

Following vaccination, the mice were challenged at week 4 with a uropathogenic E. coli strain UTI89 via transurethral catheter inoculation at a dose of 5×10⁷ CFUs per mouse.

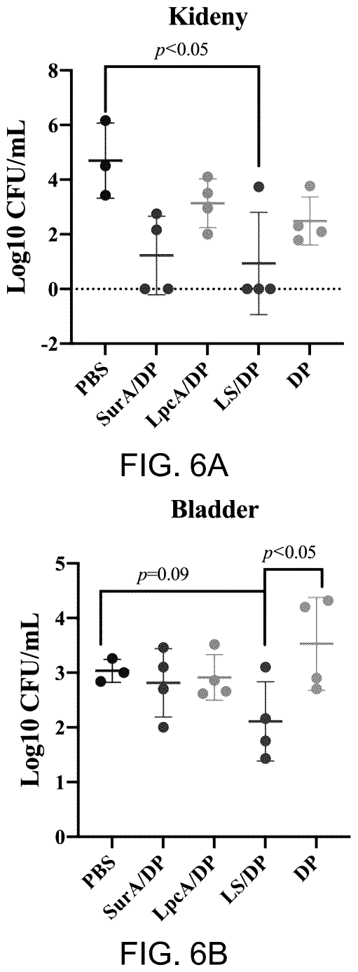

At week 1 post-challenge, bacterial loads in the bladder and kidneys were detected. Procedure: the bladder and kidneys were aseptically removed from the mice and homogenized in a tissue grinder; 1 mL of PBS buffer was added, followed by serial dilutions; and then 0.1 mL of aliquots from each dilution gradient were inoculated onto an LB agar plate. The plate was incubated in an incubator at 37° C. for 12 h, followed by CFU counting. Results: the subunit vaccine LS/DP of the present invention significantly reduced bacterial loads in the organs of immunized mice compared to both PBS and adjuvant DP control groups, as shown in FIG. 6.

To the claims:

1. A uropathogenic E. coli recombinant protein composition LS, consisting of a recombinant protein LpcA and a recombinant protein SurA, wherein the recombinant protein LpcA has an amino acid sequence shown in SEQ ID NO: 2, and the recombinant protein SurA has an amino acid sequence shown in SEQ ID NO: 4.

2. The uropathogenic E. coli recombinant protein composition LS of claim 1, wherein a mass ratio of the recombinant protein LpcA to the recombinant protein SurA is 1:1.

3. (canceled)

4. A use of the uropathogenic E. coli recombinant protein composition LS of claim 1 for preparing a vaccine for preventing uropathogenic E. coli-induced urinary tract infections.

5. A preparation method for the uropathogenic E. coli recombinant protein composition LS of claim 1, comprising the following steps:
1) performing PCR amplification on LpcA and SurA genes, and respectively inserting the LpcA and SurA genes into multiple cloning sites of a cloning vector pET30a(+) to construct recombinant vectors pET30a(+)-LpcA and pET30a(+)-SurA;
2) transforming the recombinant vectors into E. coli to obtain a strain expressing the fusion recombinant proteins expressing the fusion recombinant proteins LpcA and SurA, and utilizing the strain to express the recombinant proteins LpcA and SurA; and
3) purifying the recombinant proteins LpcA and SurA.

6. The method of claim 5, wherein the step 1) specifically comprises:
(1) based on gene sequences of LpcA GeneID: 949134, 579 bp and SurA GeneID: 944812, 1287 bp from E. coli str. K-12 in GenBank, along with restriction sites of the cloning vector, designing upstream and downstream primers containing endonucleases: SurA F and SurA R as well as LpcA F and LpcA R respectively, a nucleotide sequence of the SurA F is as shown in SEQ ID NO: 5, a nucleotide sequence of the SurA R is as shown in SEQ ID NO: 6, a nucleotide sequence of the LpcA F is as shown in SEQ ID NO: 7, and a nucleotide sequence of the LpcA R is as shown in SEQ ID NO: 8;
(2) extracting DNA from a uropathogenic E. coli strain UTI89 as a template, performing the PCR amplification on the gene sequences of LpcA and SurA using the primers obtained in the step (1), and purifying LpcA and SurA PCR amplification products by gel extraction for later use;
(3) extracting a pET30a(+) plasmid: amplifying an E. coli strain containing the pET30a(+) plasmid to 5 mL, and extracting the pET30a(+) plasmid using a plasmid extraction kit; and
(4) digesting the LpcA and SurA PCR amplification products obtained from the step (2) and the pET30a(+) plasmid extracted from the step (3) with EcoRI and HindIII restriction endonucleases; purifying the digested LpcA and SurA PCR amplification products and the digested pET30a(+) plasmid through PCR product purification and gel extraction; ligating the purified LpcA gene and the pET30a(+) plasmid as well as the purified SurA gene and the pET30a(+) plasmid using a T4 DNA ligase; and transforming ligation products into an E. coli DH5a, picking positive clones through an antibiotic plate screening, extracting plasmids, and performing sequencing identification to obtain the recombinant vectors pET30a(+)-LpcA and pET30a(+)-SurA.

7. The method of claim 5, wherein the step 2) specifically comprises:
(a) transforming the successfully constructed plasmid vectors pET30a(+)-LpcA and pET30a(+)-SurA respectively into an E. coli expression strain BL21 (DE3) to obtain a recombinant protein-preserving strain;
(b) subjecting the recombinant protein-preserving strain obtained in the step (a) to activation, shaking amplification culture, induction-shaking culture, and centrifugation in sequence to obtain a bacterial cell of the recombinant protein-preserving strain; and
(c) resuspending the bacterial cell obtained in the step (b) in a PB buffer, performing sonication in an ice bath and centrifugation, collecting a supernatant and a pellet separately, and analyzing the supernatant and the pellet separately by polyacrylamide gel electrophoresis to assess protein expression profiles.

8. The method of claim 5, wherein the step 3) specifically comprises:
(A) performing large-scale amplification on the strain expressing the fusion recombinant proteins LpcA and SurA, resuspending a harvested bacterial cell in a PB buffer, performing sonication in an ice bath and centrifugation, collecting a supernatant containing the recombinant proteins LpcA and SurA, and performing sterilization by filtration for later use; and
(B) taking purified LpcA and SurA from the supernatant obtained in the step (A) for affinity chromatography and elution to obtain the recombinant proteins LpcA and SurA.

9. The method of claim 8, wherein the affinity chromatography and the elution in the step (B) specifically comprise: first, rinsing an empty chromatography column with sterile water to ensure a smooth flow, adding 1 mL of a nickel-charged metal affinity chromatography medium (Ni-NTA) along an inner wall of the column, and after equilibrating the medium with 10 mL of imidazole binding buffer, loading the supernatant containing the recombinant proteins LpcA and SurA onto the medium for binding; then, washing the column with 10 mL of an imidazole binding buffer and 10 mL of an imidazole wash buffer in sequence, and performing a gradient elution using 50 mM, 100 mM, 200 mM, and 300 mM imidazole elution buffers; and performing elution using a 200-300 mM imidazole elution buffer to obtain the recombinant protein LpcA and performing elution using the 200-300 mM imidazole elution buffer to obtain the recombinant protein SurA, and after protein quantification, storing the recombinant proteins LpcA and SurA for later use.

10. A use of the uropathogenic E. coli recombinant protein composition LS of claim 2 for preparing a vaccine for preventing uropathogenic E. coli-induced urinary tract infections.


SEQUENCE LISTING (8 sequences)

SEQ ID NO: 1 - DNA, 579 bp, synthetic construct
  source 1..579: mol_type = other DNA
atgtaccaggatcttattcgtaacgaactgaacgaagcggcggaaacgctggctaactttttaaaagatgacgccaatattcacgccattcagcgcgcggcggtcctgttagcagacagctttaaagccggtggcaaagtgctttcctgcggcaacggcggttcccattgcgacgctatgcactttgccgaagagttgaccggtcgctaccgtgaaaaccgtccgggctacccggcgattgctatttctgacgttagtcatatttcctgcgtcggtaatgatttcggtttcaatgatattttctcccgctacgttgaagcggtaggtcgcgaaggcgatgtactgctggggatctccacctccggtaactctgcaaacgtgatcaaagcgatcgcagcggcgcgtgagaagggaatgaaagtgatcaccctgaccggtaaagacggcggcaaaatggctggcacggcggatatcgaaattcgcgtaccgcactttggttatgccgaccgcattcaggagattcacattaaagtgatccatatcctgatccagttgattgaaaaagagatggttaagtaa

SEQ ID NO: 2 - protein, 192 aa, synthetic construct
MYQDLIRNELNEAAETLANFLKDDANIHAIQRAAVLLADSFKAGGKVLSCGNGGSHCDAMHFAEELTGRYRENRPGYPAIAISDVSHISCVGNDFGFNDIFSRYVEAVGREGDVLLGISTSGNSANVIKAIAAAREKGMKVITLTGKDGGKMAGTADIEIRVPHFGYADRIQEIHIKVIHILIQLIEKEMVK

SEQ ID NO: 3 - DNA, 1287 bp, synthetic construct
  source 1..1287: mol_type = other DNA
atgaagaactggaaaacgctgcttctcggtatcgccatgatcgcgaataccagtttcgctgccccccaggtagtcgataaagtcgcagccgtcgtcaataacggcgtcgtgctggaaagcgacgttgatggattaatgcagtcggtaaaactgaacgctgctcaggcaaggcagcaacttcctgatgacgcgacgctgcgccaccaaatcatggaacgtttgatcatggatcaaatcatcctgcagatggggcagaaaatgggagtgaaaatctccgatgagcagctggatcaggcgattgctaacatcgcgaaacagaacaacatgacgctggatcagatgcgcagccgtctggcttacgatggactgaactacaacacctatcgtaaccagatccgcaaagagatgattatctctgaagtgcgtaacaacgaggtgcgtcgtcgcatcaccatcctgccgcaggaagtcgaatccctggcgcagcaggtgggtaaccaaaacgacgccagcactgagctgaacctgagccacatcctgatcccgctgccggaaaacccgacctctgatcaggtgaacgaagcggaaagccaggcgcgcgccattgtcgatcaggcgcgtaacggcgctgatttcggtaagctggcgattgctcattctgccgaccagcaggcgctgaacggcggccagatgggctggggccgtattcaggagttgcccgggatcttcgcccaggcattaagcaccgcgaagaaaggcgacattgttggcccgattcgttccggcgttggcttccatattctgaaagttaacgacctgcgcggcgaaagcaaaaatatctcggtgaccgaagttcatgctcgccatattctgctgaaaccgtcgccgatcatgactgacgaacaggcccgtgtgaaactggaacagattgctgctgatatcaagagtggtaaaacgacttttgctgccgcagcgaaagagttctctcaggatccaggctctgctaaccagggcggcgatctcggctgggctacaccagatattttcgatccggccttccgtgacgccctgactcgcctgaacaaaggtcaaatgagtgcaccggttcactcttcattcggctggcatttaatcgaactgctggatacccgtaatgtcgataaaaccgacgctgcgcagaaagatcgtgcataccgcatgctgatgaaccgtaagttctcggaagaagcagcaagctggatgcaggaacaacgtgccagcgcctacgttaaaatcctgagcaactaa

SEQ ID NO: 4 - protein, 428 aa, synthetic construct
MKNWKTLLLGIAMIANTSFAAPQVVDKVAAVVNNGVVLESDVDGLMQSVKLNAAQARQQLPDDATLRHQIMERLIMDQIILQMGQKMGVKISDEQLDQAIANIAKQNNMTLDQMRSRLAYDGLNYNTYRNQIRKEMIISEVRNNEVRRRITILPQEVESLAQQVGNQNDASTELNLSHILIPLPENPTSDQVNEAESQARAIVDQARNGADFGKLAIAHSADQQALNGGQMGWGRIQELPGIFAQALSTAKKGDIVGPIRSGVGFHILKVNDLRGESKNISVTEVHARHILLKPSPIMTDEQARVKLEQIAADIKSGKTTFAAAAKEFSQDPGSANQGGDLGWATPDIFDPAFRDALTRLNKGQMSAPVHSSFGWHLIELLDTRNVDKTDAAQKDRAYRMLMNRKFSEEAASWMQEQRASAYVKILSN

SEQ ID NO: 5 - DNA, 29 bp, synthetic construct
  source 1..29: mol_type = other DNA
cgcggatccatgtaccaggatcttattcg

SEQ ID NO: 6 - DNA, 27 bp, synthetic construct
  source 1..27: mol_type = other DNA
ccggaattccttaaccatctctttttc

SEQ ID NO: 7 - DNA, 34 bp, synthetic construct
  source 1..34: mol_type = other DNA
cgtgaattcatgaagaactggaaaacgctgcttc

SEQ ID NO: 8 - DNA, 32 bp, synthetic construct
  source 1..32: mol_type = other DNA
tcgaagcttagttgctcaggattttaacgtag